(19) United States
(12) Patent Application Publication
(10) Pub. No.: US 2003/0157505 A1
(43) Pub. Date: Aug. 21, 2003

(54) Novel substance library and supramolecular complexes prepared therewith

(75) Inventors: Christian Miculka, Wiesbaden (DE); Norbert Windhab, Hattersheim (DE); Albert Eschenmoser, Kusnach (CH); Gerhard Quinkert, Glashutten (DE)

Correspondence Address: PILLSBURY WINTHROP, LLP, P.O. BOX 10500, MCLEAN, VA, 22102, US

(73) Assignee: Aventis Research & Technologies Deutschland GmbH & Co. KG

(21) Appl. No.: 10/198,601

(22) Filed: Jul. 19, 2002

Related U.S. Application Data
(63) Continuation of application No. 09/180,651, filed on Jan. 14, 1999, now abandoned.
Application No. 09/180,651 is a 371 of international application No. PCT/EP97/02387, filed on May 9, 1997.

(30) Foreign Application Priority Data
May 14, 1996 (DE) 19619373.7

(51) Int. Cl. C12Q 1/68; C07H 21/04

(52) U.S. Cl. 435/6; 536/23.1

(57) ABSTRACT

The present invention relates to a substance library, to a process for the preparation thereof, to a process for the preparation of supramolecular complexes using this substance library and to the use of the supramolecular complexes prepared by means of the substance library, and to the use of the substance library itself.

### DESCRIPTION

The present invention relates to a substance library, to a process for the preparation thereof, to a process for the preparation of supramolecular complexes using this substance library and to the use of the supramolecular complexes prepared by means of the substance library, and to the use of the substance library itself.

Combinatorial strategies are important approaches in the search for novel active substances, especially in respect of finding lead structures and optimization thereof: there is simultaneous and usually automated synthesis of ensembles of structurally related compounds; the mixtures resulting thereby (called libraries) contain hundreds, thousands or even millions of individual compounds, each in a small amount. If the activity of one component in the mixture is detected by screening, the subsequent work of the chemist is restricted to determining the identity, because, after all, the synthesis protocol is known.

Whereas initial substance libraries were mainly of molecules with a linear constitution, such as peptides [K. S. Lam, S. E. Salmon, E. M. Hersh, V. J. Hruby, W. M. Kazmierski, R. J. Knapp, Nature 1991, 354, 82-84], interest is now centered in particular on “small” molecules which are important in the area of active substances, such as heterocycles [L. A. Thompson, J. A. Ellman, Chem. Rev. 1996, 96, 555-600]. The aim is to generate molecular diversity in order to speed up the finding of lead structures and optimization thereof.

The characteristic of combinatorial chemistry hitherto is that the synthesis takes place under kinetic control and that the variation by synthesis is separated from the selection. This relates to the in vitro evolution of RNA aptamers [J. R. Lorsch, J. W. Szostak, Nature 1994, 371, 31-36.] just as much as to the search for receptors using combinatorial methods [Y. Cheng, T. Suenaga, W. C. Still, J. Am. Chem. Soc. 1996, 118, 1813-1814], in which case two short peptide libraries were assembled on a steroid framework and irreversibly linked thereto.

It is an object of the present invention by providing a new type of substance library to increase by orders of magnitude, compared with substance libraries hitherto, the number of binding sites for ligands or substrate molecules investigated for their binding properties by means of a substance library, by reversible combination of, in each case, two or more identical or different molecular species present in the substance library, the intention being that the combination of the molecular species present in the substance library take place only in the presence of the substrate molecule to be investigated, via the appropriate binding interactions with the molecular species.

It is another object of the present invention to provide supramolecular complexes which arise through combination of the molecular species present in the substance library and through the binding interactions with the substrate molecule to be investigated.

Substance libraries of this type, and supramolecular complexes of this type ought to be suitable for producing medicinal active substances, active substances for crop protection, catalysts or for diagnosing diseases.

The object stated at the outset is achieved by a substance library obtainable by coupling different or identical molecular species, which are preferably present in a substance library, to a molecular pairing system.

It is possible by means of the substance library according to the present invention to prepare supramolecular complexes by exposing the substance library to an interaction with a substrate, identifying and, where appropriate, isolating the supramolecular complex formed thereby.

The present invention accordingly also relates to the provision of a supramolecular complex prepared in this way, which is suitable, for example, for producing medicinal substances, active substances for crop protection, catalysts, for diagnosing diseases and for producing corresponding diagnostic kits.

In the same way, the precursor of these supramolecular complexes, mainly the substance library according to the invention, is also suitable for producing medicinal substances, active substances for crop protection, catalysts and for diagnosing diseases, including the production of corresponding diagnostic kits.

The general terms mentioned above or used hereinafter for explaining the invention and in the claims are defined below.

Molecular species: for example molecules with a linear constitution such as peptides, in particular proteins, peptoids, linear oligo- or polysaccharides, nucleic acids and their analogs or, for example, monomers such as heterocycles, in particular nitrogen heterocycles, or molecules not with a linear constitution, such as branched oligo- or polysaccharides or else antibodies.

Supramolecular complex: produced by association of two or more molecular species which are held together by non-covalent forces.

Pairing systems: supramolecular systems of non-covalent interactions which are characterized by selectivity, stability and reversibility and whose properties are preferably influenced thermodynamically, such as, for example, by temperature, pH, concentration. Examples are preferably pyranosyl-RNA, CNA, DNA, RNA, PNA.

Interactions are preferably hydrogen bonds, salt bridges, stacking, metal liganding, charge-transfer complexes and hydrophobic interactions.

Substance library: ensemble of compounds of different structures, preferably oligomeric or polymeric peptides, peptoids, saccharides, nucleic acids, esters, acetals or monomers such as heterocycles, lipids, steroids.

Substrate: molecules, preferably medicinal substances and active substances for crop protection, metabolites, physiological messengers, derivatives of lead structures, substances which are produced, or produced to an increased extent, in the human or animal body in the event of pathological changes, transition state analogs or else peptides, in particular proteins, peptoids, linear oligo- or polysaccharides, nucleic acids and their analogs, or, for example, monomers such as heterocycles, in particular nitrogen heterocycles, or molecules not with a linear constitution, such as branched oligo- or polysaccharides or else antibodies, and substance libraries, also sites of action of drugs, preferably receptors, voltage-dependent ion channels, transporters, enzymes and biosynthetic units of microorganisms.

Transition state analogs: synthetic molecular species which are structurally similar to the assumed transition state of a chemical reaction but are, in contrast thereto, stable.

Identification can comprise isolation or characterization of the supramolecular complex, but preferably differentiation on the basis of particular properties of the supramolecular complex of substance libraries coupled to pairing systems and substrate, preferably different chromatographic, electrophoretic, spectroscopic or signal (labeling) behavior by comparison with uncomplexed species or by covalent (chemical) attachment of the species involved in the complex formation.

CNA: cyclohexylnucleooligoamide; represents a synthetic variant of the DNA structure in which the phosphate-sugar backbone is replaced by 2-(3-aminocyclohexyl)ethanoic acid units, with the units being linked together in the manner of a peptide, and the 3-aminocyclohexyl substituents each being provided with a nucleobase in position 4.

The substance library according to the present invention is preferably distinguished by the pairing system consisting of one longer and two shorter base strands, with the two shorter strands being complementary to the longer strand at different points but not being complementary to one another, and with a gap of at least one base remaining between the short strands in the event of base-pairing with the longer strand, while at least one base remains unpaired in the region of this gap corresponding to the size thereof on the longer strand, with those bases on each of the two shorter strands which are located at the start and at the end of the pairing gap being linked by a linker to one molecular species in each case, while at least one of the unpaired bases in the longer strand is linked by a linker to a molecular species.

Peptides with different properties can be reversibly combined in a controlled manner to give groups of two or three, for example by linkage to pairing oligonucleotide ends. This means that, owing to the large number of possible combinations, orders of magnitude more different binding sites are generated in the experiment than peptides have been synthesized.

Implementation of the principle of combinatorial variation and selection under thermodynamic control, and linkage thereof, represents an elementary technological leap in combinatorial methods: the relevant receptor is formed by combination only in the presence of the substrate.

This receptor thus reacts to the presence of the substrate: if the latter is equated with an antigen, the present system can be regarded analogously as an “artificial immune system”.

Nature has produced a remarkable number of molecules which carry out the complex processes of the living organisms—from the immune response and catalysis to signal transmission. For this it has recourse to a broad combinatorial library of precursor molecules and checks these for the required properties. Probably the most important example of this strategy is the immune system which is able to generate an enormous molecular diversity and scan the latter for receptors with high affinity and selectivity for foreign antigens. The combination of molecules which intrinsically bind only weakly, if at all, to a stable binding complex is also a principle which is widespread in nature (heteromers [D. E. Clapham, Nature 1996, 379, 297-299]) and whose significance for use in combinatorial chemistry has not yet been recognized.

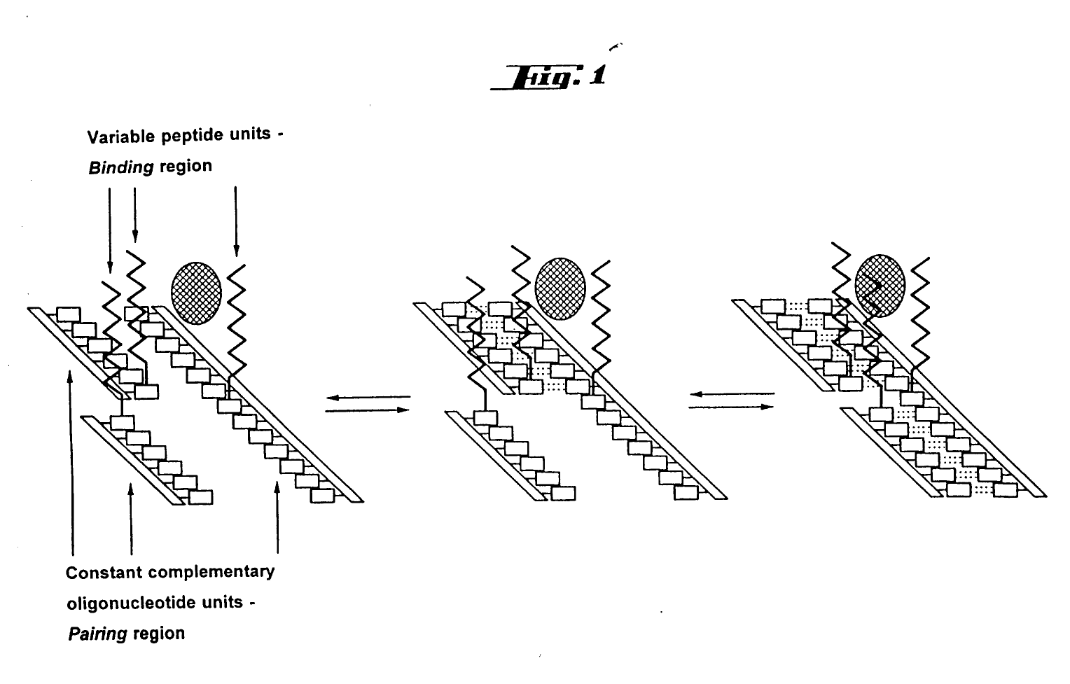

FIG. 1 shows diagrammatically the structure and the process of formation of such a receptor: a short peptide chain (as library) is covalently attached via a linker unit to the middle building block of an oligonucleotide composed, for example, of 13 monomer building blocks. An analogous procedure is applied to the two end units of the short oligonucleotides consisting of 6 monomer units.

If these three units are then offered to the substrate (ellipse), a competition for the best binding of the peptide moieties to the substrate starts: the pairing between the oligonucleotides ensures approach of the peptide moieties in space. The reversibility of the individual steps is crucial, resulting in exchange of the individual peptide regions until the most stable complex has been found. This process, which takes place under thermodynamic control, corresponds to an automatic experimental molecular modeling. In fact, in such an experiment, all the possible transiently occurring combinations of the three libraries is subjected to the selection. This exchange process is temperature-dependent, i.e. exchange of the individual strands is more frequent at higher temperature, but, at the same time, the interactions of the peptide moieties with the substrate become weaker.

After freezing of the equilibrium, covalent crosslinking of the pairing partners, isolation and decomplexation, the receptor is obtained in free form.

A process for the preparation of supramolecular complexes has therefore been designed and comprises coupling compound libraries to pairing systems.

Supramolecular complexes which have been prepared under thermodynamic control by the processes below and selected coupled under thermodynamic control are used when molecules or molecular regions are to be recognized. The advantage is that the libraries, which are always the same, are able very quickly in combination to solve increasingly novel selection problems.

These are, in particular:

a) Molecular recognition of biologically relevant substances, i.e. diagnosis. The development of diagnostic methods in particular must keep up with the variety of substrates to be recognized, such as metabolites or, for example, continually mutating pathogens, so that the benefits of this process are obvious.

b) Molecular recognition of biologically relevant substances, i.e. drug design. The described process generates highly selective supramolecular complexes which themselves act as active substances or as models for the development of active substances in that, for example, they bind to, and thus stimulate or block, pharmacological receptors. On the other hand, the supramolecular complexes act as receptors in the development of active substances, because a profile of interactions of the active substances can be drawn up with their aid.

c) Thermodynamically controlled constitution of catalytically active supramolecular complexes, for example by offering transition state analogs as substrates in the sense of the use as catalytic antibodies [L. C. Hsieh-Wilson, X.-D. Xiang, P. G. Schultz, Acc. Chem. Res. 1996, 29, 164-170].

### PROCEDURAL EXAMPLE 1

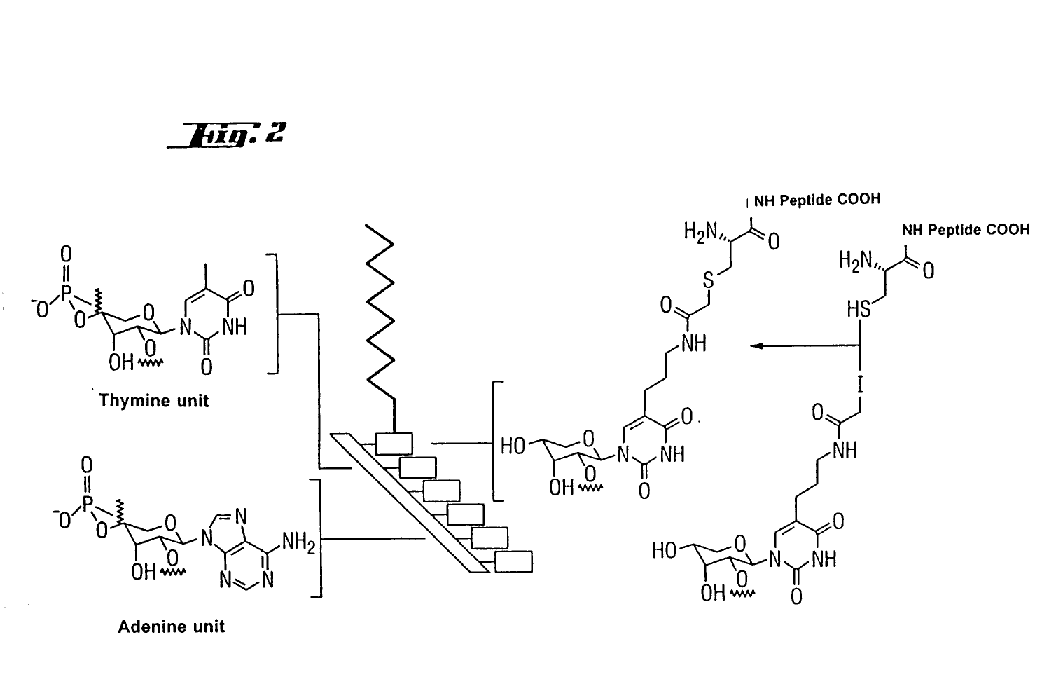

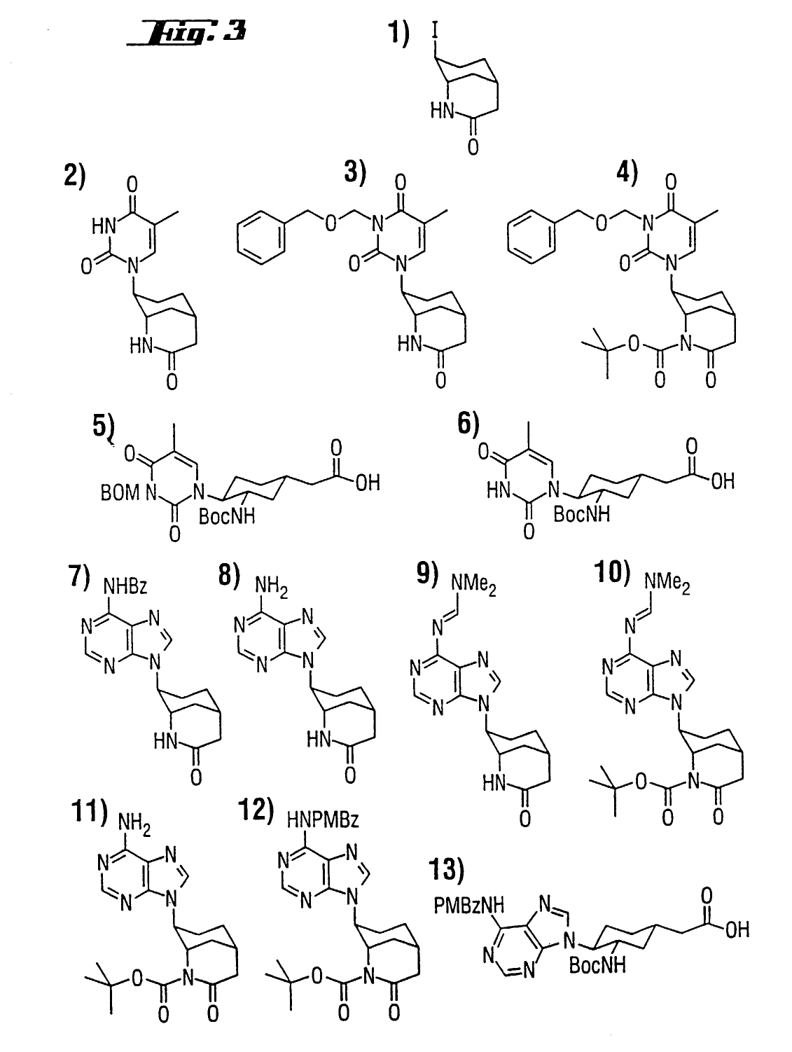

Pyranosyl-RNA is used as pairing system (see FIG. 2), the preparation and properties of which are well known [S. Pitsch, S. Wendeborn, B. Jaun, A. Eschenmoser, Helv. Chim. Acta 1993, 76, 2161-2183]. Starting from D-ribose and the nucleobases adenine and thymine, phosphoramidites capable of coupling are prepared as described therein, and the required hexamer and tridecamer sequences are prepared using an oligonucleotide synthesizer. The tridecamer has the sequence 2′-AATTAAT*ATATAT, one hexamer has the sequence 2′-T*TAATT-4′, and the other hexamer has the sequence 2′-ATATAT*-4′, where T* is the linker nucleotide building block. The linker nucleotide building block is synthesized by methods known from the literature starting from the uracil nucleoside: iodination [W.-W. Sy, Synth. Comun. 1990, 20, 3391-3394], reaction with propargyl phthalimide, and hydrogenation [K. J. Gibson, S. J. Benkovic, Nucleic Acids Res. 1987, 15, 6455-6467] affords the required building block. Hydrazinolysis and iodoacetylation of the oligonucleotide takes place as described in the literature [T. Zhu, S. Stein, Bioconjugate Chem. 1994, 5, 312-315]. Tetrapeptides are prepared as compound libraries starting from commercially obtainable amino acid monomers using a multiple peptide synthesizer, providing an N-terminal cysteine residue as linker unit. The library is divided into three portions and allowed to react in aqueous buffered solution at room temperature in each case with the two hexamer sequences and the tridecamer sequence to give the required conjugates, which are purified by reverse phase chromatography [T. Zhu, S. Stein, Bioconjugate Chem. 1994, 5, 312-315]. Pairing of the complementary units is detected on the basis of the decrease in the UV extinction in the pairing experiment.

### PROCEDURAL EXAMPLE 2

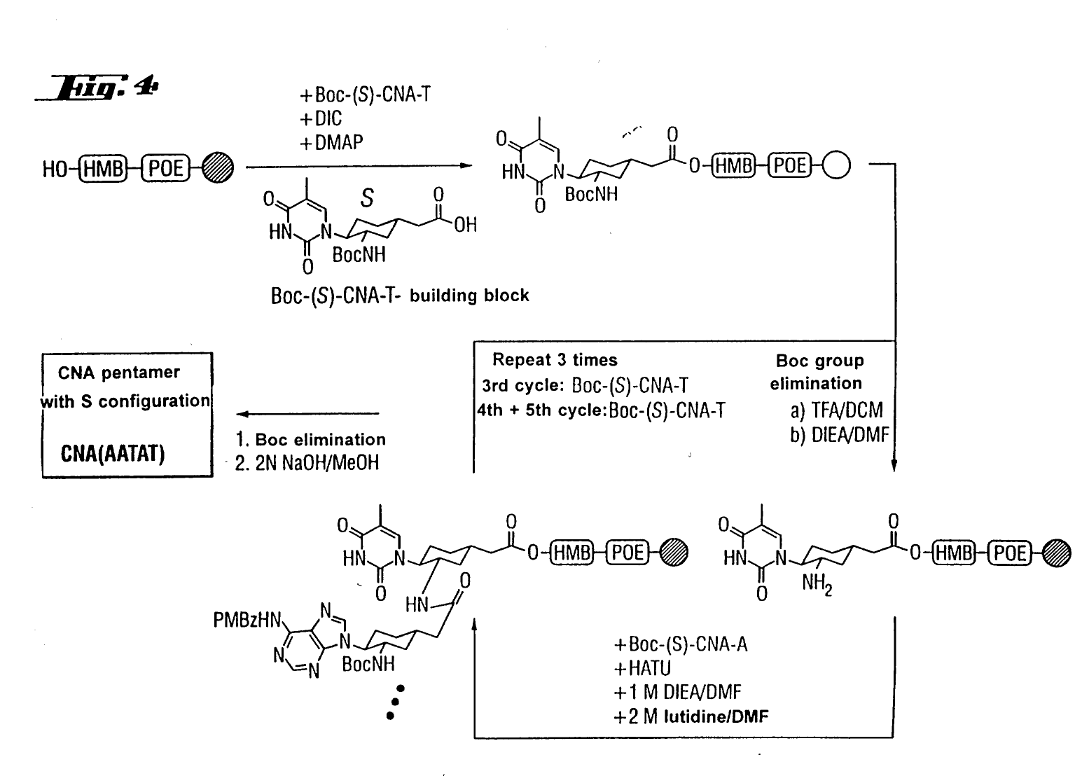

Solid-Phase Synthesis of a CNA Pentamer (FIG. 4)

The CNA oligomer was synthesized in analogy to the peptide or oligonucleotide synthesis, by stepwise incorporation of individual building blocks on a solid phase. For this the necessary reagents were added in excess and unreacted amounts were removed again by simple washing steps. The polymeric support used was a polyoxyethylene (POE)/polystyrene copolymer (Tentagel S HMB, 0.23 mmol/g), which has good swelling properties both in aqueous solution and in organic solvents.

The aminoethyl functionalities of the polymer were derivatized with a hydroxymethylbenzoyl (HMB) linker; the loading with the first building block took place using a 5-fold excess by the symmetrical anhydride method (addition of 2.5 eq of DIC) and by adding the acylation catalyst DMAP (2.5 eq) over the course of 20 h in DCM. The resulting loading amounted to 0.17 mmol/g. The Boc protective group of the amino functionality was eliminated with 50% TFA in DCM, and then the resin was neutralized with 1 M DIEA/DMF. The subsequent cycles consisted of repetitive coupling of the next monomer and elimination of the Boc protective group. The couplings took place after preactivation of the monomer building block (3 eq.) with the activation reagent HATU (3 eq.) in DMF (40 μl) and with addition of 1 M DIEA/DMF (6 eq.) and 2 M lutidine/DMF (12 eq.). The coupling times were 3-4 h at room temperature. After four coupling cycles, the N-terminal Boc protective groups were eliminated and the pentamer was cleaved off the resin with 2 N NaOH in methanol over the course of 15 min. The elimination solution was removed from the resin by filtration and kept at 55° C. for 2 h. Neutralization with 2 N HCl was followed by purification with C18 RP-HPLC (Hibar prepacked column 250-4, RP-18, 5 μm) with gradient elution (1 ml/min) from 10% to 40% B in 30 min (solvents A: water+0.1% TFA, B: acetonitrile+0.1% TFA). The synthesis of CNA(AATAT) was carried out with 10 mg (1.7 μmol) of Tentagel-HMB resin which was loaded with (S)-CNA-thymine monomer building block. All the CNA building blocks have the S configuration. The sequence reading from left to right corresponds to the way of writing from the N to the C terminus usual in peptide chemistry.

CNA(AATAT): HPLC: Rt=14.30 min; UV: λmax=264 nm; ESI-MS: [M+H]⁺_{calc} 1362.0, [M+2H]²⁺_{calc} 681.0; [M+H]⁺ₑₓₚ 1361.8, [M+2H]²⁺ₑₓₚ 681.5.

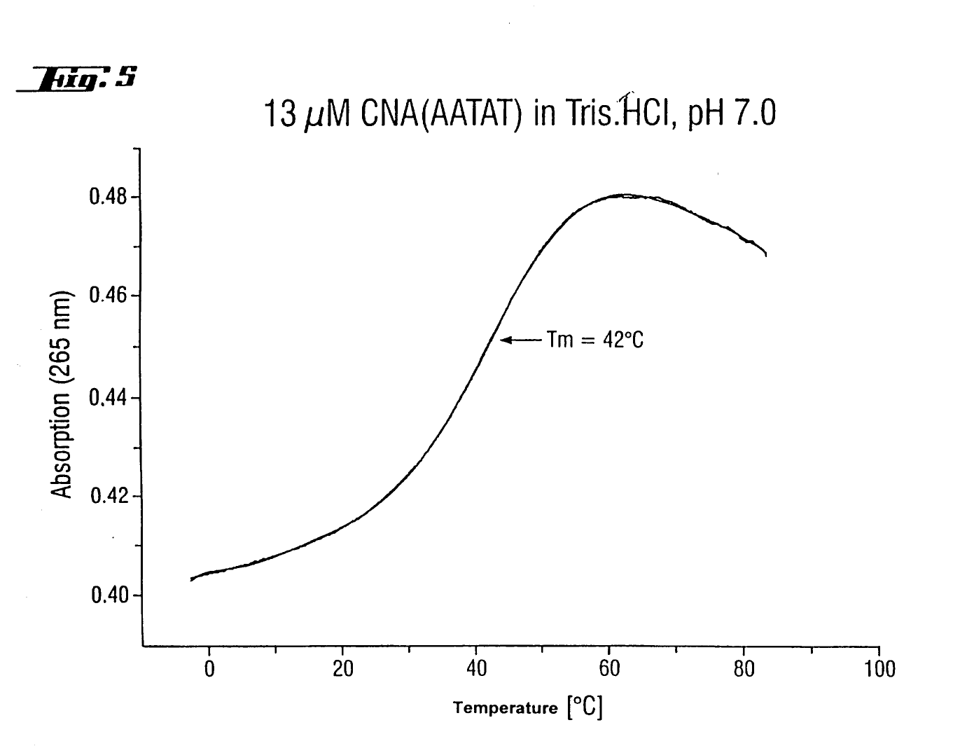

Desalting of the CNA pentamer CNA(AATAT) was followed by measurement, in a Perkin Elmer Lambda 2 UV-VIS spectrometer, of the temperature-dependent extinctions at 265 nm with six different concentrations over a range from 0 to 80° C. (1.5-50 μM in TrisHCl buffer at pH 7.0). The first derivative of these reversible, sigmoid transition plots yields the melting temperature (Tm=42° C. at 13 μM) (FIG. 5).

Solid-Phase Synthesis of a Peptide-CNA Conjugate

The CNA pentamer described above was, before elimination from the resin, extended by a dipeptide library at the N terminus. The sequence is XO-CNA(AATAT). X represents a mixed position in which the five L-amino acids alanine, aspartic acid, leucine, lysine and serine are varied. O represents a defined position, with O=L-lysine being chosen for this sublibrary. Coupling of Boc-Lys(Fmoc)-OH to the Boc-deprotected CNA pentamer CNA(AATAT) took place after preactivation of the amino acid building block (6 eq.) with the activation reagent HATU (6 eq.) in DMF and with addition of 1 M DIEA/DMF (7 eq.). The coupling time was 3 h. Introduction of the X position took place by the split resin method. After elimination of the N-terminal Boc protective group, 100 μl of DMF:DCM (1:1) were added to the amount of resin (5 mg), and the mixture was divided into five portions of equal size, each of 20 μl. Coupling of the individual amino acids took place in parallel in separate reaction vessels with about 1 mg of oligomer-resin in each case. The individual Boc-protected amino acids, Boc-Ala-OH, Boc-Asp(OFm)-OH, Boc-Leu-OH, Boc-Ser-OH and Boc-Lys(Fmoc)-OH were coupled in 50-fold excess after preactivation with HATU (50 eq.) and with addition of 1 M DIEA/DMF (100 eq.) at room temperature for 3 h. After elimination of the N-terminal Boc protective groups, the Fmoc protective groups were removed with 40% piperidine/DMF (20 min). The peptide-CNA oligomer conjugates were cleaved off the resin in each case with 2 N NaOH in methanol over the course of 15 min. The elimination solution was removed from the resin by filtration and kept at 55° C. for 2 h. Neutralization with 2 N HCl was followed by purification with C18-RP-HPLC (Hibar ready acid 250-4, RP-18, 5 μm) with gradient elution (1 ml/min) from 10% B to 40% B in 30 min (solvents A: water+0.1% TFA, B: acetonitrile+0.1% TFA).

HPLC: Ala-Lys-CNA(AATAT)Rt=15.47 min Asp-Lys-CNA(AATAT)Rt=15.30 min Leu-Lys-CNA(AATAT)Rt=16.08 min Lys-Lys-CNA(AATAT)Rt=15.34 min Ser-Lys-CNA(AATAT)Rt=15.29 min

ESI-MS: Ala-Lys-CNA(AATAT) [M+H]⁺_{calc} 1561.6; [M+H]⁺ₑₓₚ 1561.4 Asp-Lys-CNA(AATAT) [M+H]⁺_{calc} 1605.7; [M+H]⁺ₑₓₚ 1605.3 Leu-Lys-CNA(AATAT) [M+H]⁺_{calc} 1603.8; [M+H]⁺ₑₓₚ 1603.4 Lys-Lys-CNA(AATAT) [M+H]⁺_{calc} 1619.3; [M+H]⁺ₑₓₚ 1619.0 Ser-Lys-CNA(AATAT) [M+H]⁺_{calc} 1577.7; [M+H]⁺ₑₓₚ 1578.7

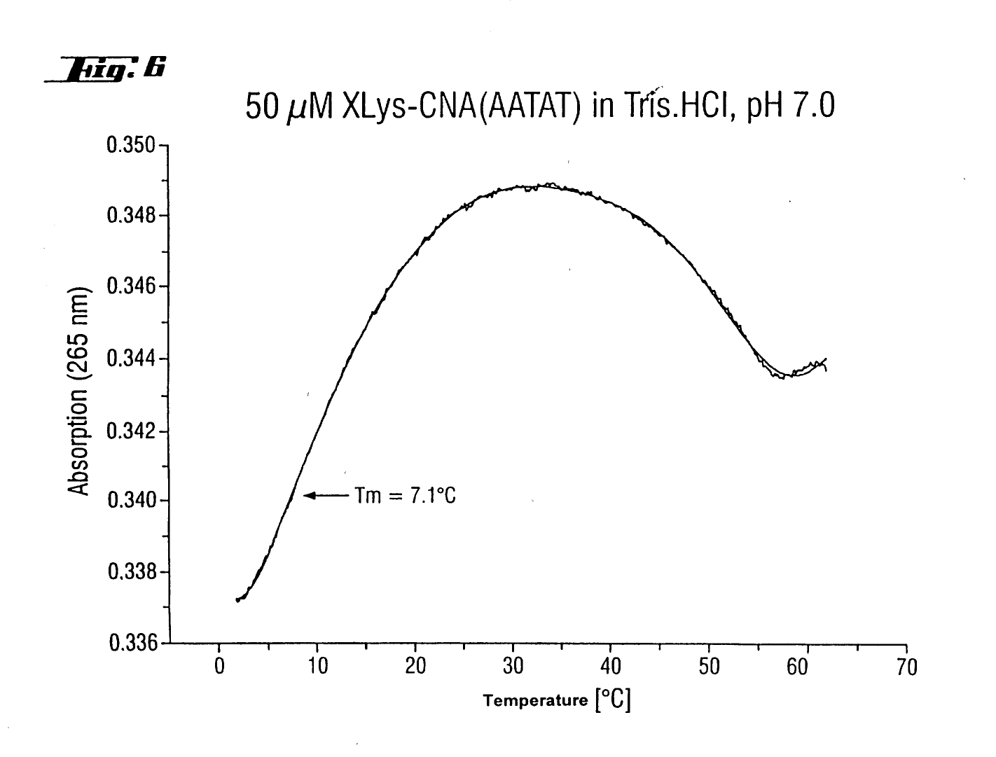

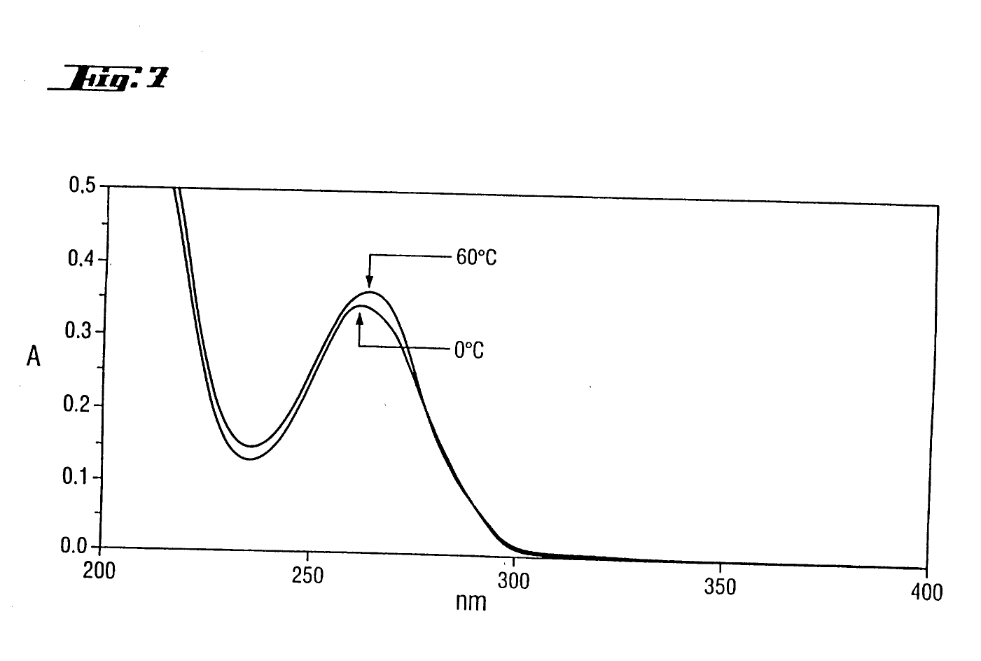

After characterization of the individual components, the HPLC fractions were combined. Desalting of the peptide library CNA oligomers XLys-CNA(AATAT) was followed by measurement, in a Perkin Elmer Lambda 2 UV-VIS spectrometer, of the temperature-dependent extinctions at 265 nm at 50 μM over a range of 0-60° C. (in TrisHCl buffer at pH 7.0). The first derivative of this temperature plot yields the melting temperature (Tm=7° C. at 50 μM) (FIG. 6). The UV spectra of XLys-CNA(AATAT) at 0° C. and 60° C. differ in qualitatively the same way as the pentamer without library CNA(AATAT). The wavelengths of the absorption maximum shifts from 261.4 nm (E=0.3427) at 0° C. to 263.8 nm (E=0.3626) at 60° C. and thus proves the existence of the supramolecular complexes, i.e. an equilibrating combinatorial library (FIG. 7).

The meanings in this context are

Boc tert-Butyloxycarbonyl

DMC Dichloromethane

DIC Diisopropylcarbodiimide

DIEA Diisopropylethylamine

DMAP Dimethylaminopyridine

DMF Dimethylformamide

Fmoc Fluorenylmethyloxycarbonyl

HATU O-[7-Azabenzotriazol-1-yl]-1,1,3,3-tetramethyluroniumhexafluorophosphate

TFA Trifluoroacetic acid

We claim:

1. A substance library obtainable by coupling different or identical molecular species to a molecular pairing system.

2. A substance library as claimed in claim 1, wherein the molecular species are present in a substance library.

3. A substance library as claimed in claim 1, wherein the pairing system is a nucleic acid.

4. A substance library as claimed in claim 3, wherein the pairing system is a DNA.

5. A substance library as claimed in claim 3, wherein the pairing system is an RNA.

6. A substance library as claimed in claim 3, wherein the pairing system is a pyranosyl-RNA.

7. A substance library as claimed in claim 1, wherein the pairing system is a PNA.

8. A substance library as claimed in claim 1, wherein the pairing system is a CNA.

9. A substance library as claimed in claim 1, wherein the molecular species are molecules with a linear constitution.

10. A substance library as claimed in claim 9, wherein the molecular species are peptides.

11. A substance library as claimed in claim 9, wherein the molecular species are peptoids.

12. A substance library as claimed in claim 1, wherein the molecular species are oligo- or polysaccharides.

13. A substance library as claimed in claim 9, wherein the molecular species are nucleic acids or analogs thereof.

14. A substance library as claimed in claim 1, wherein the molecular species are monomers.

15. A substance library as claimed in claim 3, wherein the pairing system consists of one longer and two shorter base strands, with the two shorter strands being complementary to the longer strand at different points but not being complementary to one another, and with a gap of at least one base remaining between the short strands in the event of base-pairing with the longer strand, while at least one base remains unpaired in the region of this gap corresponding to the size thereof on the longer strand, with those bases on each of the two shorter strands which are located at the start and at the end of the pairing gap being linked by a linker to one molecular species in each case, while at least one of the unpaired bases in the longer strand is linked by a linker to a molecular species.

16. A process for the preparation of a supramolecular complex, which comprises exposing a substance library as claimed in claim 1 to an interaction with a substrate, and identifying and, where appropriate, isolating the supramolecular complex formed thereby.

17. The process as claimed in claim 16, wherein the substrate is a peptide.

18. The process as claimed in claim 16, wherein the substrate is a peptoid.

19. The process as claimed in claim 16, wherein the substrate is an oligo- or polysaccharide.

20. The process as claimed in claim 16, wherein the substrate is a nucleic acid or analog thereof.

21. The process as claimed in claim 16, wherein the substrate is a medicinal substance.

22. The process as claimed in claim 16, wherein the substrate is an active substance for crop protection.

23. The process as claimed in claim 16, wherein the substrate is an analog of one or more molecules in the transition state of a chemical reaction.

24. The process as claimed in claim 16, wherein the substrate is a metabolite.

25. The process as claimed in claim 16, wherein the substrate is a physiological messenger.

26. The process as claimed in claim 16, wherein the substrate is a substance which is produced, or produced to an increased extent, in the human or animal body in the event of pathological changes.

27. The process as claimed in claim 16, wherein the substrate is sites of action of drugs, preferably receptors, voltage-dependent ion channels, transporters, enzymes and biosynthetic units of microorganisms.

28. A supramolecular complex obtainable by a process as claimed in claim 16.

29. A method of using a supramolecular complex as claimed in claim 28 for the preparation of a medicinal active substance.

30. A method of using a supramolecular complex as claimed in claim 28 for the preparation of an active substance for crop protection.

31. A method of using a supramolecular complex as claimed in claim 28 for the preparation of a catalyst.

32. A method of using a supramolecular complex as claimed in claim 28 for the diagnosis of diseases.

33. A method of using a supramolecular complex as claimed in claim 28 for the production of a diagnostic kit.

34. A diagnostic kit comprising a supramolecular complex as claimed in claim 28.

35. A method of using a substance library as claimed in claim 1 for the diagnosis of diseases.

36. A method of using a substance library as claimed in claim 1 for the production of a diagnostic kit.

37. A method of using a substance library as claimed in claim 1 for the preparation of a catalyst.

38. A process for the preparation of a substance library as claimed in claim 1, which comprises coupling molecular species, which may be different or identical, to a pairing system.

39. A cyclohexylnucleooligoamide comprising aminocyclohexylethanoic acid units.
